(19) United States
(12) Patent Application Publication
(10) Pub. No.: US 2007/0123926 A1
(43) Pub. Date: May 31, 2007

(54) Pre-curved guiding catheter with mechanically actuated occluder for embolic protection

(75) Inventors: Ghaleb A. Sater, Acton, MA (US); Jeffrey J. Witts, North Reading, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/287,880

(22) Filed: Nov. 28, 2005

(51) Int. Cl. A61M 29/00

(52) U.S. Cl. 606/194; 604/96.01; 606/200

(57) ABSTRACT

A guiding catheter includes an elongate shaft with a central bore and a mechanically expandable occluder mounted about the distal end of the shaft. The occluder comprises a flexible membrane supported by an expandable tubular frame. The guiding catheter has a pre-formed curve adjacent the distal end of the shaft. A sheath disposed about the catheter is selectively slidable along the catheter shaft to expand the occluder into sealing engagement with the wall of an artery to provide hemostasis in the artery. Methods of using the guiding catheter are also disclosed.

### FIELD OF THE INVENTION

The present invention relates generally to an intraluminal guiding catheter used in a medical procedure, and more particularly, to a guiding catheter with embolic protection by proximal occlusion.

### BACKGROUND OF THE INVENTION

A stenosis, or narrowing of a blood vessel such as a coronary artery may comprise a hard, calcified substance and/or a softer thrombus material. There have been numerous therapeutic procedures developed for the treatment of stenosis in a coronary artery. One of the better-known procedures is percutaneous transluminal coronary angioplasty (PTCA). According to this procedure, the narrowing in the artery can be reduced by positioning a dilatation balloon across the stenosis and inflating the balloon to re-establish acceptable blood flow through the artery. Additional therapeutic procedures may include stent deployment, atherectomy, and thrombectomy, which are well known and have proven effective in the treatment of such stenotic lesions.

The therapeutic procedure starts with the introduction of a guiding catheter into the cardiovascular system from a convenient vascular access location, such as through the femoral artery in the groin area or other locations in the arm or neck. The guiding catheter is advanced through the arteries until its distal end is located near the stenosis that is targeted for treatment. During PTCA, the distal end of the guiding catheter is typically inserted only into the ostium, or origin of the coronary artery. A guidewire is advanced through a central bore in the guiding catheter and positioned across the stenosis. A therapy device, such as balloon dilatation catheter, is then slid over the guidewire until the dilatation balloon is properly positioned across the stenosis. The balloon is inflated to dilate the artery. To help prevent the artery from re-closing, a physician can implant a stent inside the artery. The stent is usually delivered to the artery in a compressed shape on a stent delivery catheter and expanded by a balloon to a larger diameter for implantation against the arterial wall.

Recently, a variety of devices have been developed to address atheroembolization, which is the obstruction of blood vessels by stenotic debris released during interventional catheterization therapies such as those mentioned above. Distal protection devices (DPDs) such as filters and occluders represent one class of intravascular devices that can be used to prevent atheroembolization. A filter mounted on a guidewire or a catheter may be positioned distally of a stenotic lesion to capture and remove potentially embolic debris without causing hemostasis. Alternatively, an occluder device may be positioned distally of a stenotic lesion to temporarily stop the flow of blood, including any stenotic debris that may have become entrained in the blood. The contaminated blood is aspirated from the treated area before the occluder device is collapsed to permit resumption of blood flow.

Occlusion devices may also be placed proximally of a stenotic lesion to provide so-called proximal protection. Proximal occlusion devices may be used alone to prevent atheroembolization, or they may be used in conjunction with a distal occluder to form an isolated treatment chamber about the lesion to be treated. Preliminary deployment of a proximal occlusion device may be advantageous in preventing atheroembolization because advancing a treatment catheter into a tight stenosis can dislodge particulate debris; even before the stenosis is being opened.

One type of guiding catheter that may be utilized is described in U.S. Patent Application Publication No. 2002/0026145 A1 entitled “Method and Apparatus for Emboli Containment” to Bagaoisan et al. (“Bagaoisan”). Typical of most guiding catheters, the Bagaoisan catheter is pre-curved at the distal end to set and hold a supporting position in the vasculature while the therapeutic catheter crosses and treats the lesion. Additionally, the Bagaoisan catheter includes an expandable sealing balloon disposed around the guiding catheter distal end that, when appropriately positioned, may be inflated to provide embolic protection by proximal occlusion.

Another type of guiding catheter that may be utilized is described in U.S. Pat. No. 6,544,276 to Azizi. In addition to a pre-curved distal end, the guiding catheter in the '276 patent teaches a self-expanding sealing member disposed around the guiding catheter distal end. A sliding sleeve encases the self-expanding sealing member and may be retracted to release same.

Known occluder devices typically employ an inflatable occlusion balloon with its attendant expansion apparatuses, which may make the system cumbersome to prepare and use. Additionally, multi-catheter systems used to form isolated treatment chambers may be complex to use when it is desirable for the physician to work quickly to minimize the duration of hemostasis. Furthermore, having a sleeve slide over a self-expanding sealing member can increase the overall size of a guiding catheter. Thus, a need exists for a guiding catheter having a low-profile atheroembolization prevention system that may be activated and deactivated simply and quickly during interventional catheterization procedures. Other desirable features and characteristics of the present invention will become apparent from the subsequent detailed description and the appended claims taken in conjunction with the accompanying drawings.

### BRIEF SUMMARY OF THE INVENTION

The invention provides a guiding catheter with embolic protection by artery occlusion. The guiding catheter includes an elongate shaft having a central bore and a pre-curved region adjacent the distal end. A sheath is slidably disposed about the shaft. A tubular frame is attached between the distal ends of the shaft and the sheath. The tubular frame is responsive to longitudinal movement between the ends of the frame to transform between a collapsed configuration and an expanded configuration, the expanded configuration having a centrally located major diameter. A non-porous flexible membrane extends along the frame between the frame proximal end and the major diameter such that, when the frame is in the expanded configuration, the major diameter of the frame is apposed to the artery wall and the membrane occludes the artery.

A method is disclosed for using the inventive guiding catheter with mechanically actuated occluder for embolic protection. The method includes providing a guiding catheter having the embodiment described above; inserting the guiding catheter into the vascular system of the patient and positioning the flexible membrane proximal to the stenotic lesion to be treated; and moving the sheath along the shaft to expand the frame and sealing membrane into sealing engagement with the wall of the artery to provide proximal occlusion of blood flow.

In other embodiments of the invention, the method may also include: inserting a therapeutic device through the central bore of the guiding catheter; positioning the therapeutic portion of the therapeutic device across the stenosis; and treating the stenosis with the therapeutic device.

### BRIEF DESCRIPTION OF THE DRAWINGS

The following drawings are illustrative of particular embodiments of the invention and therefore do not limit its scope. They are presented to assist in providing a proper understanding of the invention. The drawings are not to scale and are intended for use in conjunction with the explanations in the following detailed descriptions. Like reference numerals denote like elements in the drawings, wherein;

FIG. 1 is a side view of a distal region of a guiding catheter in accordance with the invention, shown with an occluder in a collapsed configuration;

FIG. 2 is a side view of a distal region of the guiding catheter shown in FIG. 1, shown with the occluder in an expanded configuration

FIG. 3 is a transverse cross-sectional view of the guiding catheter shown in FIG. 2, taken along line 2-2;

FIG. 3 is a side view of a distal portion of the guiding catheter of FIG. 1, shown with the sealing member in a contracted configuration;

FIG. 4 illustrates a guiding catheter in accordance with the invention, shown deployed in the cardiovascular system of a patient; and

FIGS. 5-8 illustrate the use of the inventive guiding catheter in a diseased vessel during a typical angioplasty procedure.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of protection against atheroembolization during treatment of blood vessels such as the coronary, carotid and renal arteries, the invention may also be used in any other passageways where it is deemed useful to provide temporary occlusion to block fluid flow. Furthermore, there is no intention to be bound by any expressed or implied theory presented in the preceding technical field, background, brief summary or the following detailed description.

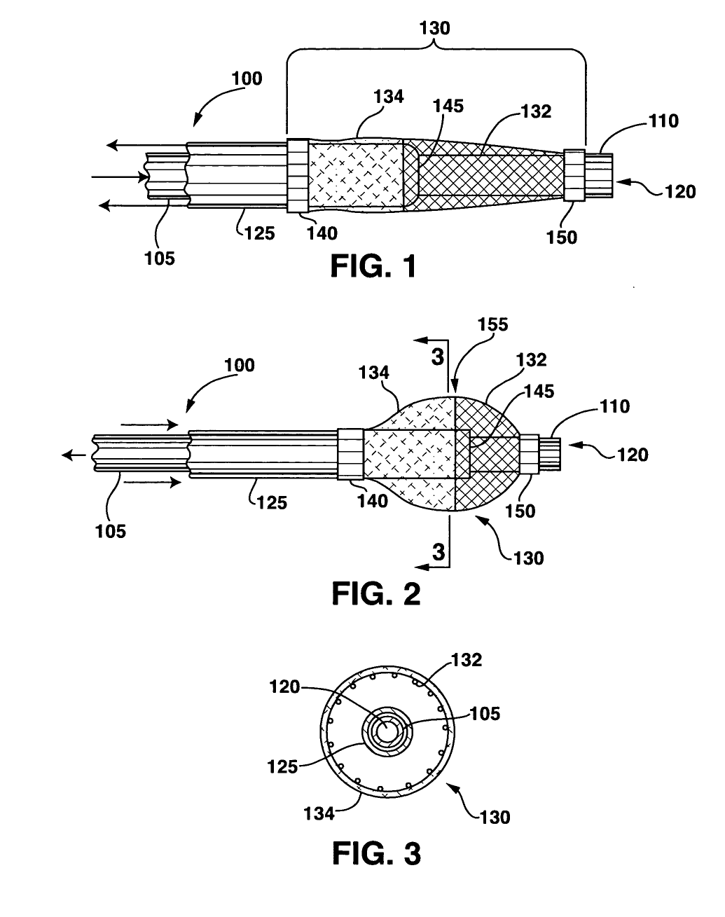

FIGS. 1 and 2 illustrate a distal region of one embodiment of guiding catheter 100, including elongate shaft 105 with distal end 110, which may comprise an optional soft tip. Bore 120 extends through shaft 105 between open proximal and distal ends, has a low-friction surface and is sized and shaped to receive and direct there through a variety of treatment devices such as guidewires and/or therapeutic devices including, but not limited to balloon catheters or stent delivery systems. Elongate sheath 125 is slidably disposed about shaft 105, terminating adjacent shaft distal end 110.

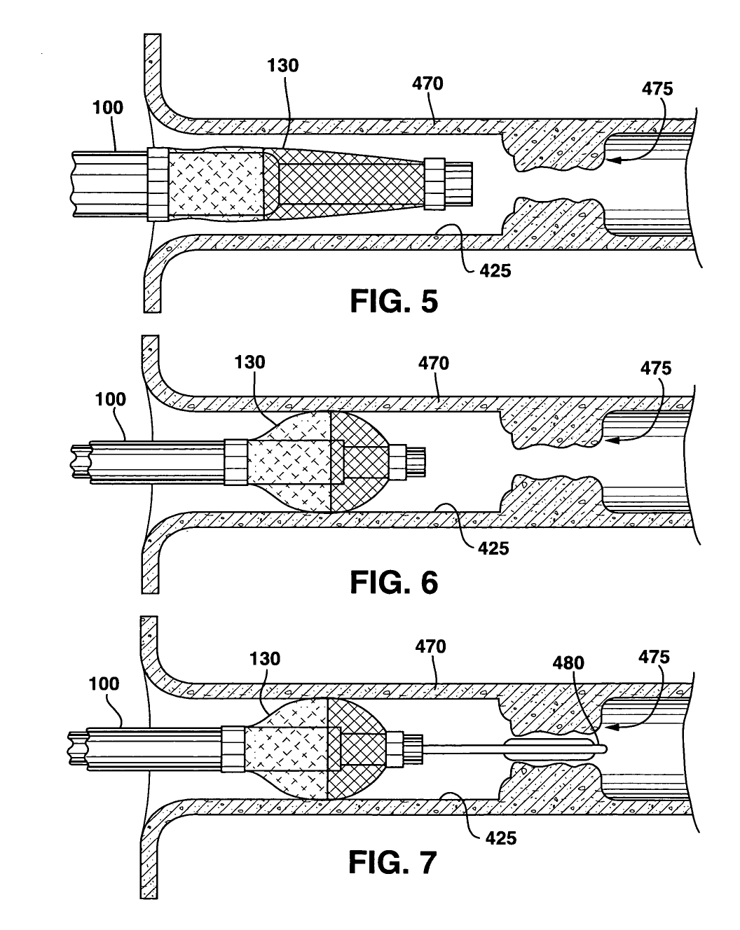

Occluder 130 is mounted about a distal region of catheter 100 and includes tubular frame 132 clingingly encased by, or impregnated by impermeable flexible membrane 134. Frame proximal end 140 is affixed adjacent sheath distal end 145 and frame distal end 150 is affixed adjacent shaft distal end 110. In FIG. 1, tubular frame 132 is shown in a collapsed configuration. In FIG. 2, tubular frame 132 is shown in an expanded configuration having a broadest transverse section, or major diameter 155. Sliding sheath 125 proximally or distally along shaft 105 translates frame ends 140, 150 apart or together causing transformation of frame 132 between expanded and collapsed configurations. In the unrestricted expanded configuration, major diameter 155 is greater than the diameter of a branch vessel lumen being intubated, such that frame 132 causes membrane 134 to seal against the lumen wall and cause temporary stasis in the vessel, as will be described in further detail below in conjunction with FIGS. 5-8.

Catheter shaft 105 is a flexible tube that is designed to advance through a patient's vasculature to remote arterial locations without buckling or undesirable bending. As is well known to those of skill in the art, catheter shaft 105 includes a pre-formed distal curve that can provide enhanced “backup support” as therapeutic catheters are advanced through bore 120 of guiding catheter 100 and across a stenosis. Any one of a number of pre-formed curve shapes may be incorporated into guiding catheter 100, such as Judkins-type or Amplatz-type curves, as non-limiting examples. Curve 160 may be pre-formed utilizing various known methods including, but not limited to, the method disclosed in U.S. Pat. No. 5,902,287 entitled “Guiding Catheter and Method of Making Same.”

Catheter shaft 105 may be constructed of one or more flexible biocompatible materials, including, but not limited to, polyethylene, polypropylene, polyurethane, polyesters, or polyethylene block amide copolymer. Catheter shaft 105 may also include a layer of braided filaments that resist kinking and enhance longitudinal transmission of rotation. To further aid in advancing guiding catheter 100 through the patient's vasculature, it may be desirable to vary the stiffness of catheter shaft 105 by varying the braid pitch, by varying the properties of materials used in construction, or by combining both techniques.

Bore 120 of guiding catheter 100 may provide a slippery interior surface for reducing frictional forces between the interior surface and devices that may be moved through bore 120. In one exemplary embodiment, the interior surface is provided with a slippery coating, such as a silicone compound or a hydrophilic polymer. In another exemplary embodiment, the interior surface includes a liner formed from a slippery material. Those with skill in the art may appreciate that any one of numerous low-friction, biocompatible materials such as, for example, fluoropolymers (e.g. PTFE, FEP), polyolefins (e.g. polypropylene, high-density polyethylene), or polyamides, may be used for bore 120.

Sheath 125 may comprise flexible biocompatible materials such as those mentioned above with respect to shaft 105. Furthermore, to provide a small overall diameter of guiding catheter 100, sheath 125 may comprise thin-walled thermoset polyimide tubing, which has sufficient stiffness to provide precise manual actuation of occluder 130 by pushing or pulling sheath 125 relative to shaft 105, as indicated by the force vectors in FIGS. 1 and 2.

Tubular frame 132 may comprise braided filaments or alternatively, an expandable array of struts formed by making parallel slots a solid-walled tube (not shown). The braid filaments or tubing of frame 132 may be made from a high-modulus thermoplastic or thermo-set plastic, nitinol (TiNi), stainless steel or a work-hardenable super alloy comprising nickel, cobalt, chromium and molybdenum. Frame proximal and distal ends 140, 150 may be fixed to sheath 125 and catheter shaft 105, respectively, by any suitable manner known in the art, such as epoxy or cyanoacrylate adhesives. Radiopaque material may be incorporated into one or both of the adhesive bonds, either as a solid marker band or as particulate filler material in the adhesive. Frame proximal end 140 may abut or be located directly around the distal end of sheath 125. Alternatively, proximal end 140 may be spaced somewhat proximally from the distal end of sheath 125, as illustrated in FIGS. 1 and 2, to provide an intermediate step in both diameter and stiffness along the assembly.

Sealing membrane 134 has sufficient flexibility such that when it is actuated or expanded it will form a seal between sheath 125 and the inner wall of the artery or desired vessel. When membrane 134 is contracted or deactivated it will lie snugly against frame 132. Membrane 134 may be formed from an elastic material such as latex, silicone elastomer, or other viscous forms of natural and synthetic rubbers such as butadiene/acrylonitride copolymers, copolyesters, ethylene vinylacetate (EVA) polymers, ethylene/acrylic copolymers, ethylene/propylene copolymers, polyalkylacrylate polymers, polybutadiene, polybutylene, polyethylene, polyisobutylene, polyisoprene, polyurethane, styrenebutadiene copolymers, and styrene-ethylene/butylene-styrene.

Alternatively, membrane 134 may be formed from an inelastic material that is thin, flexible and foldable, such as polyamide, polyethylene, polyethylene terephthalate, polyolefin, polypropylene, or polyvinyl chloride. As shown in FIG. 3, sealing membrane 134 may be attached around the outer surface of tubular frame 132 using any suitable manner known in the art, such as adhesive bonding or heat bonding. The proximal end of membrane 134 may be sealingly affixed about frame proximal end 140, or sealingly affixed to sheath 125 adjacent to frame proximal end 140. Alternatively, membrane 134 may be impregnated into the interstices of tubular frame 132, using known techniques such as thermoplastic molding or solvent dipping or casting.

Membrane 134 covers a proximal region of tubular frame 132 by extending distally from frame proximal end 140 at least as far as major diameter 155. The distal region of tubular frame 132 that is not covered by membrane 134 allows unrestricted fluid flow through the interstices of frame 132, thus permitting rapid transformation of occluder 130 between the collapsed and expanded configurations.

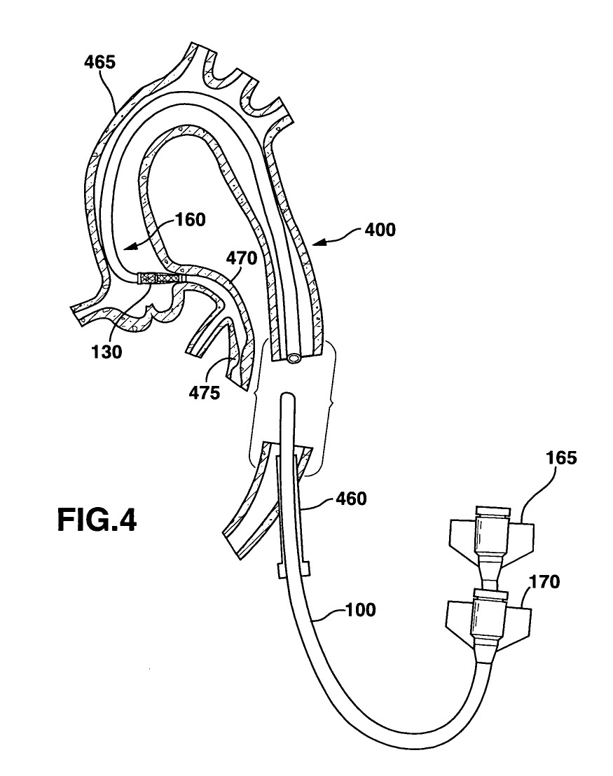

As shown in FIG. 4, connector fitting 165 is coupled to, and provides a functional access port at the proximal end of guiding catheter 100. Fitting 165 is attached to shaft 105 and has a central opening in communication with bore 120 to allow passage of therapeutic devices there through. Connector fitting 165 may be made of metal or of a hard polymer (e.g. medical grade polycarbonate, polyvinyl chloride, acrylic, acrylonitrile butadiene styrene (ABS), or polyamide) that possesses the requisite structural integrity, as is well known to those of skill in the art.

Control fitting 170 is coupled to the proximal end of sheath 125 and has a central opening to allow shaft 105 to slide there through. Fitting 170 provides an enlarged component for the clinician to manually grasp when sliding sheath 125 along shaft 105 to actuate occluder 130. Optionally, fitting 170 may include a mechanism (not shown) for temporarily locking shaft 105 and sheath 125 in their relative longitudinal positions that define either the expanded or collapsed configurations of occluder 130. Fitting 170 may be made of the same or similar material as those mentioned above with respect to connector fitting 165.

An exemplary method of using guiding catheter 100 will now be described. FIG. 4 illustrates guiding catheter 100 positioned within patient's vascular system 400 for use with a therapeutic device. The clinician confirms that occluder 130 is in the compressed configuration and inserts the distal end of guiding catheter 100 through introducer sheath 460 into vascular system 400, typically through a femoral artery in the groin area. Guiding catheter 100 is advanced through aorta 465 until the distal end of the catheter is located in the ostium of targeted branch artery 470. In the example shown, branch artery 470 is a patient's left coronary artery.

If the clinician elects to use proximal occlusion during the intervention, then the distal end of guiding catheter 100 is inserted into artery 470 until occluder 130 is substantially within artery 470, as illustrated in FIG. 5. At least the portion of occluder 130 that will become, when in the expanded configuration, major diameter 155 is positioned distal to the ostium. The clinician manually separates connector fitting 165 and control fitting 170 to actuate occluder 130, thus transforming it from the collapsed configuration to the expanded configuration, which provides a seal between occluder 130 and vessel wall 425 and create hemostasis within artery 470, as illustrated in FIG. 6. Connector fitting 165 and control fitting 170 are manually separated according to the clinician's preference: Shaft 105 may be held stationary in artery 470 while sheath 125 is advanced, or sheath 125 may be held stationary in artery 470 while shaft 105 is withdrawn.

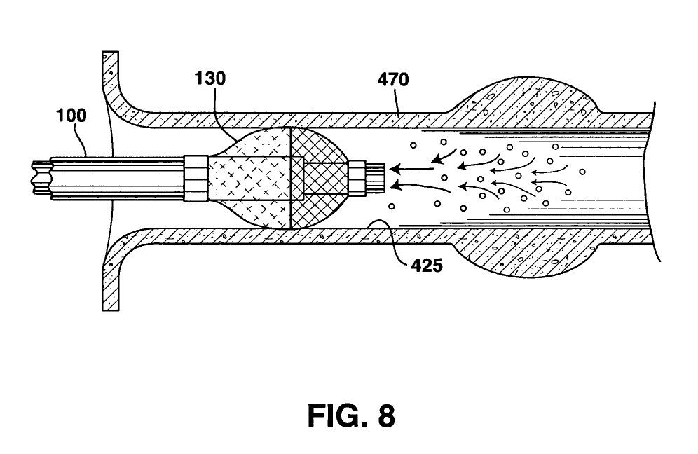

A therapeutic device, such as balloon dilatation catheter 480, including a dilatation balloon, is advanced through bore 120 until the balloon reaches a desired position within stenosis 475, as illustrated in FIG. 7. The dilatation balloon is then inflated to dilate stenosis 475. Balloon dilatation catheter 480 may then be removed, and blood may be aspirated from artery 470, including any debris released during the dilation of stenosis 475 as shown in FIG. 8. Aspiration may be performed either directly into bore 120 of guiding catheter 100 or, alternatively, into an aspiration catheter (not shown), which may be advanced to the treated area within artery 470. As will be recognized by those of skill in the art, aspiration during proximal occlusion of an artery requires retrograde blood flow in artery 470. If retrograde flow through the capillary bed is insufficient to support aspiration of the potentially contaminated blood, then simultaneous flush and aspiration can be established through an aspiration catheter and guiding catheter 100. Lastly, guiding catheter 100 is withdrawn from vessel lumen 420.

While at least one exemplary embodiment has been presented in the foregoing detailed description, it should be understood that various changes can be made in the function and arrangement of elements without departing from the scope of the invention as set forth in the appended claims and the legal equivalents thereof.

What is claimed is:

1. A guiding catheter for intubation and selective embolic protection in a branch vessel in a patient, the vessel having a lumen and a lumen wall, the catheter comprising:
an elongate hollow shaft having open proximal and distal ends and a pre-curved region adjacent the shaft distal end;
an elongate sheath slidably disposed about the shaft and having a sheath distal end terminating adjacent the shaft distal end;
a tubular frame mounted about the shaft and having a frame distal end fixed adjacent the shaft distal end and a frame proximal end fixed adjacent the sheath distal end, wherein relative longitudinal movement between the ends of the frame accompanies transformation of the frame between a collapsed configuration and an expanded configuration, the expanded configuration having a centrally located major diameter; and
a non-porous flexible membrane extending along the frame between the frame proximal end and the major diameter such that, when the frame is in the expanded configuration, the major diameter is capable of being apposed to the vessel wall such that the membrane occludes the vessel.

2. The guiding catheter of claim 1, wherein the tubular frame comprises multiple braided filaments.

3. The guiding catheter of claim 1, wherein the elongate hollow shaft has a reinforcement layer.

4. The guiding catheter of claim 3, wherein the reinforcement layer comprises a tubular braid.

5. The guiding catheter of claim 1, wherein the flexible membrane is adhered over or impregnated into the tubular frame.

6. The guiding catheter of claim 1, wherein the flexible membrane is an elastic biocompatible material.

7. The guiding catheter of claim 6, wherein the elastic biocompatible material is latex, silicone elastomer, butadiene/acrylonitride copolymers, copolyesters, ethylene vinylacetate (EVA) polymers, ethylene/acrylic copolymers, ethylene/propylene copolymers, polyalkylacrylate polymers, polybutadiene, polybutylene, polyethylene, polyisobutylene, polyisoprene, polyurethane, styrenebutadiene copolymers, or styrene-ethylene/butylene-styrene.

8. The guiding catheter of claim 1, wherein the tubular frame is selectively transformable between the collapsed configuration and the expanded configuration by sliding the sheath longitudinally along the shaft.

9. The guiding catheter of claim 1 further comprising a connector fitting mounted at the shaft proximal end in communication with a bore extending between the open proximal and distal shaft ends.

10. A method of using a guiding catheter comprising:
providing a guiding catheter in accordance with claim 1;
inserting the catheter shaft distal end into a vascular system of the patient;
advancing the catheter shaft distal end to the branch vessel in the patient;
intubating the vessel with the catheter shaft distal end; and
sliding the sheath distally along the shaft to transform the tubular frame into the expanded configuration wherein the major diameter apposes the vessel wall such that the membrane occludes the vessel.

11. The method of claim 10 further comprising:
inserting a therapeutic device through the guiding catheter; and
operating the therapeutic device to treat the patient from within the branch vessel.

12. The method if claim 11, wherein the therapeutic device is an angioplasty catheter and operating the therapeutic device comprises inflating a balloon to dilate a stenosis in the vessel.

13. The method of claim 10 further comprising:
aspirating blood from the branch vessel through the catheter shaft distal end.

14. The method of claim 11 further comprising:
aspirating contaminated blood from the branch vessel through the catheter shaft distal end.

15. The method of claim 13 further comprising:
sliding the sheath proximally along the shaft to transform the tubular frame into the collapsed configuration; and
withdrawing the catheter shaft from the vascular system of the patient.
